(19) United States
(12) Patent Application Publication
(10) Pub. No.: US 2011/0077509 A1
(43) Pub. Date: Mar. 31, 2011

(54) MEASURING DEVICE AND A METHOD FOR DETERMINING TISSUE PARAMETERS

(75) Inventors: Martin Leibfritz, Muenchen (DE); Christian Evers, Kirchheim (DE)

(73) Assignee: ROHDE & SCHWARZ GMBH & CO. KG, Muenchen (DE)

(21) Appl. No.: 12/994,389

(22) PCT Filed: Jun. 2, 2009
(86) PCT No.: PCT/EP09/03920

§ 371 Date: Nov. 23, 2010

(30) Foreign Application Priority Data
Jun. 2, 2008 (DE) 10 2008 026 435.0
Dec. 22, 2008 (DE) 10 2008 064 405.6

(51) Int. Cl. A61B 5/055

(52) U.S. Cl. 600/424

(57) ABSTRACT

A measuring device provides a control device, a test-signal transmitter, a test-signal receiver and a test probe. The test probe contains at least one coaxial line. The control device controls the test-signal transmitter in such a manner that it transmits a test signal by means of the coaxial line into a given location of the tissue. The test signal is scattered by the tissue. The control device controls the test-signal receiver in such a manner that it receives the scattered test signal. The control device evaluates the received test signal. The test probe is designed in such a manner that a tissue specimen can be removed by it at the given location of the tissue.

The invention relates to a measuring device and a method for determining tissue parameters.

Biopsies are conventionally used for determining tissue parameters. However, a biopsy is always associated with tissue damage. Furthermore, the precise location for the preparation of a biopsy cannot be reliably determined.

Moreover, the measurement of tissue parameters by means of electrical signals is known. For example, WO 03/060462 A2 discloses a device for measuring electrical parameters of a tissue. In this context, a sensor is placed on a tissue. An electrical signal then impinges on the tissue. A resulting signal is measured. Information about the tissue is obtained from the transmitted signal and the received signal. The disadvantage here is that, in order to obtain relatively accurate information, a removal of the sensor and the subsequent implementation of further tests is necessary. An exact positioning at the identical location of the tissue is not possible. This results in a low accuracy.

Furthermore, a combination of a biopsy needle with a measurement of electrical parameters is known. Accordingly, WO 2005/009200 A2 discloses a device, which measures electrical properties of the tissue by means of a sensor integrated in the tip of a special biopsy needle. After the completion of the measurement, a tissue specimen can be obtained by means of the biopsy needle. However, the tissue specimen is obtained at the side of the biopsy needle. Only a low accuracy is achieved as a result of the lack of agreement between the measuring position and the biopsy position.

A measurement of electrical parameters by means of an off-tuned resonator is also known. Accordingly, WO 2006/103665 A2 discloses a sensor for tissue characterisation, which contains a resonator. In this context, the resonator is brought into contact with the tissue and off-tuned by the latter. The tissue properties are inferred from the off-tuning. The disadvantage here is that the resonator is connected only via a single supply line. Accordingly, only a low accuracy is guaranteed because of scattering. Furthermore, the resonator structures disclosed here allow only a low-accuracy measurement. In particular, as a result of the three-dimensional design of the resonator, interference is caused, which further reduces the accuracy of the measurement. Moreover, a removal of the tissue specimens is not possible here.

The invention is based upon the object of providing a device and a method for determining tissue parameters with low stress on the patient and a good accuracy of the parameters.

The object is achieved according to the invention by the features of the independent claims 1 and 7 for the measuring device, and by the features of the independent claims 13 and 16 for the method. Advantageous further developments form the subject matter of the dependent claims referring back to these claims.

A measuring device according to the invention comprises a control device, a test-signal transmitter, a test-signal receiver and a test probe. The test probe contains at least one coaxial line. The control device controls the test-signal transmitter in such a manner that it transmits a test signal by means of the coaxial line into a given location of the tissue. The test signal is scattered by the tissue. The control device controls the test-signal receiver in such a manner that it receives the scattered test signal. The control device evaluates the received test signal. The test probe is designed in such a manner that a tissue specimen can be removed by it at the given location of the tissue. Accordingly, a protective electrical measurement can be implemented, before a tissue specimen is removed. This reduces the number of tissue specimens required from the patient. Furthermore, the number of tissue specimens to be investigated is reduced.

The test probe preferably contains a biopsy needle. The coaxial line is preferably arranged within the biopsy needle. In this manner, conventional biopsy needles can be used. Furthermore, the use of very thin, flexible, favourable coaxial lines is possible.

The coaxial line is advantageously mobile within the biopsy needle. The coaxial line is preferably withdrawn to a location, at which a tissue specimen is to be removed, by at least a length of the tissue specimen. The tissue specimen preferably penetrates into the biopsy needle and is preferably fixed by the latter. Accordingly, a tissue specimen can be obtained from precisely the location of the electrical measurement. The exact length of the tissue specimen can be determined very accurately in this manner. This reduces the stress on the patient and increases the accuracy of determination of the tissue parameters.

An end of the coaxial line alternatively provides sharp edges. The coaxial line advantageously provides a rigid form. In this manner, the coaxial line can be inserted into the tissue without a supporting biopsy needle. This reduces the cost of manufacture and simplifies handling.

The coaxial line preferably contains an inner conductor, an outer conductor and a dielectric. The dielectric is preferably mobile. The dielectric is preferably withdrawn at a location, at which a tissue specimen is to be removed, by at least a length of the tissue specimen. The tissue specimen advantageously penetrates into an intermediate cavity between the outer conductor and the inner conductor and is fixed by the latter. In this manner, in spite of the simple manufacture and handling, a tissue specimen can be obtained at precisely the location of the electrical measurement.

As an alternative, the test probe contains two coaxial lines. The test-signal transmitter preferably transmits the test signal into the tissue by means of a first coaxial line. The test-signal receiver preferably receives the test signal by means of a second coaxial line. Accordingly, electromagnetic scattering into the test line can be avoided. The accuracy of the electrical measurement can be improved in this manner.

An alternative measuring device according to the invention contains a control device, a test-signal transmitter, a test-signal receiver and a test probe. The test probe contains at least two coaxial lines and a resonator circuit. A first coaxial line is connected to the resonator circuit and to the test-signal transmitter. A second coaxial line is connected to the resonator circuit and the test-signal receiver. The control device controls the test-signal transmitter in such a manner that it transmits a test signal by means of the first coaxial line to the resonator circuit. The control device controls the test-signal receiver in such a manner that it receives the scattered test signal by means of the second coaxial line from the resonator circuit. The resonance properties of the resonator circuit are influenced by tissue disposed in its proximity. The control device evaluates the received test signal. A very accurate electrical measurement can be implemented in this manner.

The resonator circuit preferably consists of a printed-circuit board with at least one strip conductor disposed on it. A simple manufacture of the resonator circuit is possible in this manner.

Three strip conductors are preferably arranged on the printed-circuit board. A first strip conductor is preferably connected in a conducting manner to the first coaxial line. A second strip conductor is preferably connected in a conducting manner to the second coaxial line. A third strip conductor is preferably disposed between the first strip conductor and the second strip conductor. The third strip conductor is advantageously connected to the first strip conductor and to the second strip conductor in a capacitive manner. A simple manufacture of the resonator circuit using a standard technology is possible in this manner with high precision of the electrical measurement.

The third strip conductor is preferably annular or rectilinear in shape. The third strip conductor advantageously determines the resonance wavelength of the resonator circuit. The length of the strip conductor is advantageously ¼ to ¾, preferably ½ of the resonance wavelength of the resonator circuit. A very accurate measurement with small space requirement of the resonator circuit is possible in this manner.

Tissue to be investigated is preferably disposed in the proximity of the third strip conductor. A strong effect of the tissue on the resonator can be achieved in this manner. This allows a very high measurement accuracy.

The test probe is preferably designed in such a manner that a tissue specimen can be removed by it at the given location of the tissue. Accordingly, in the presence of given results of the electrical measurement, a tissue specimen can be obtained from exactly the location of the measurement without further stress on the patient.

The invention is described by way of example below with reference to the drawings, in which an advantageous exemplary embodiment of the invention is presented. The drawings are as follows:

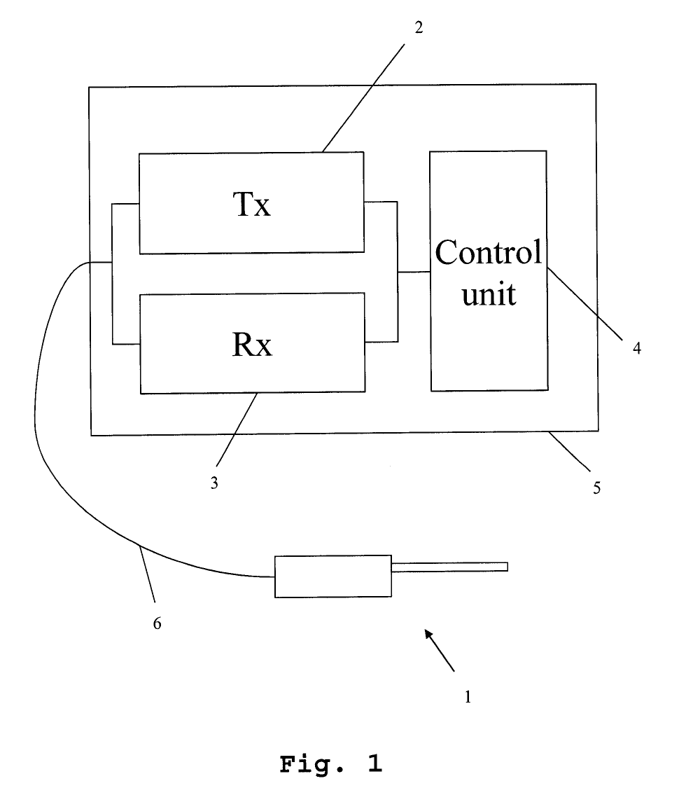

FIG. 1 shows a first exemplary embodiment of a measuring device according to the invention;

FIG. 2 shows a detail of a second exemplary embodiment of the measuring device according to the invention;

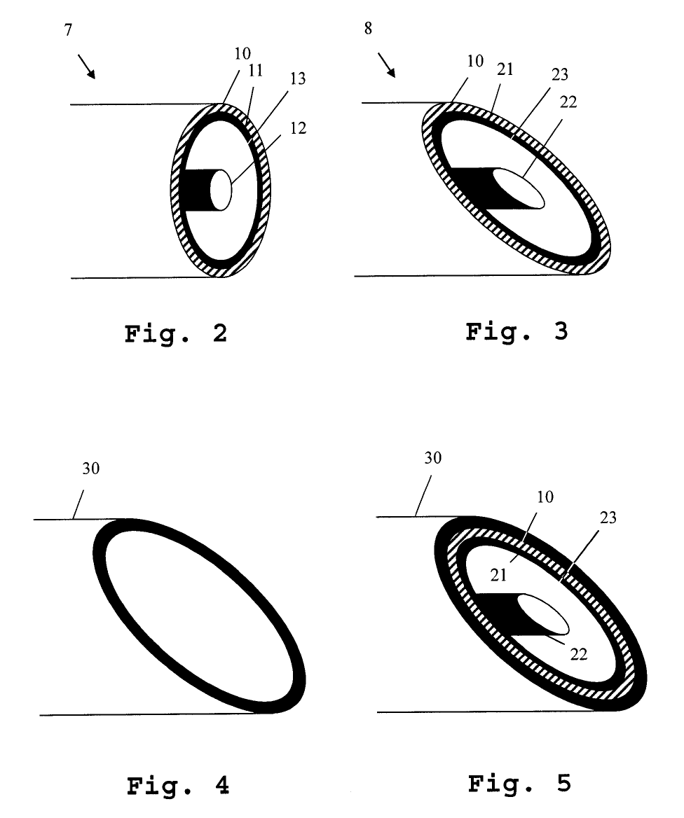

FIG. 3 shows a detail of a third exemplary embodiment of a measuring device according to the invention;

FIG. 4 shows a second detail of the third exemplary embodiment of a measuring device according to the invention;

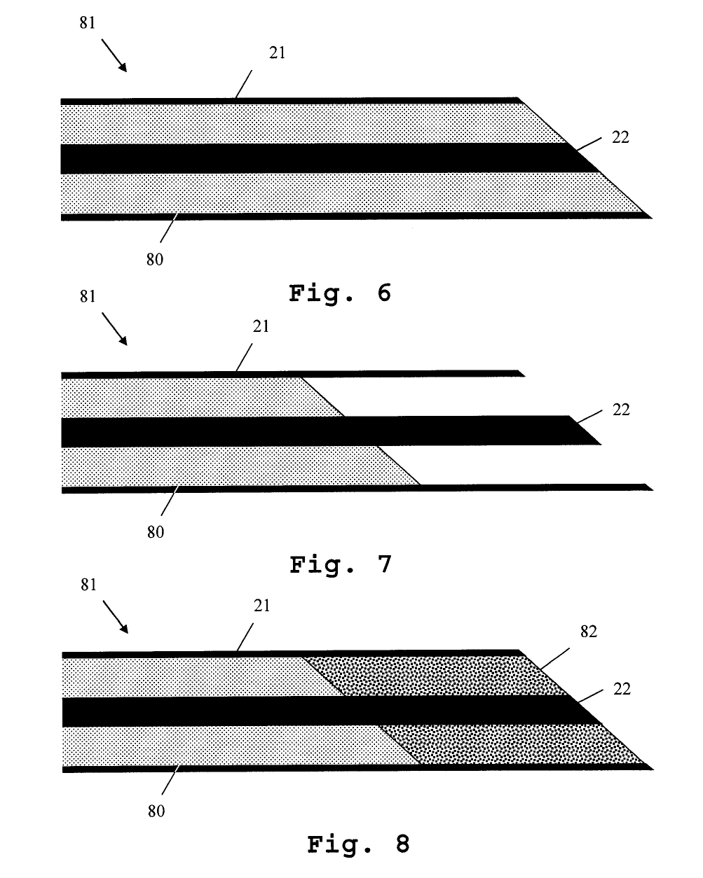

FIG. 5 shows a third detail of the third exemplary embodiment of a measuring device according to the invention;

FIG. 6 shows a first sectional view of a fourth exemplary embodiment of a measuring device according to the invention;

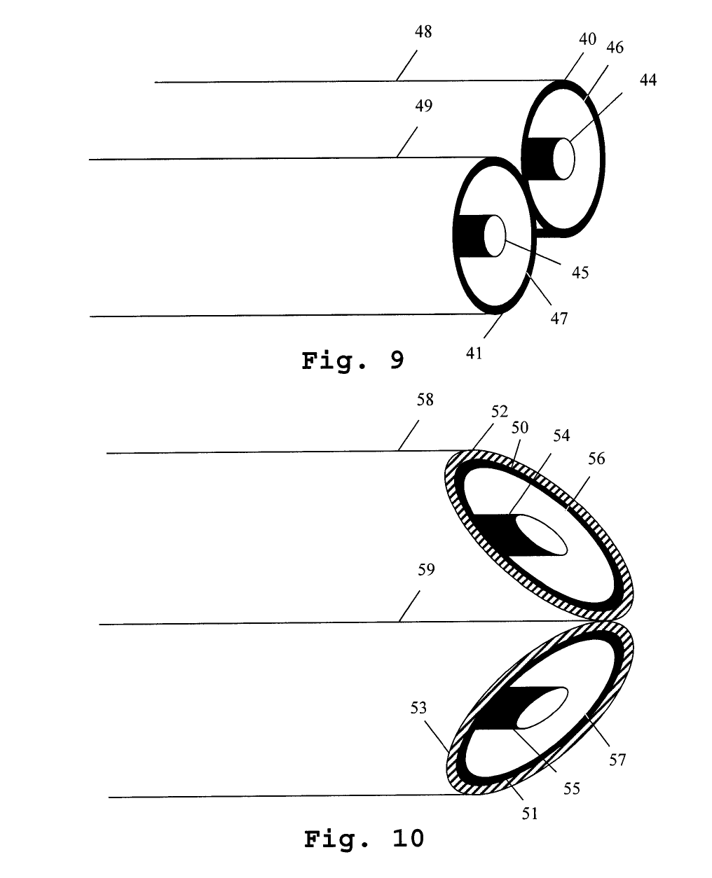

FIG. 7 shows a second sectional view of the fourth exemplary embodiment of a measuring device according to the invention;

FIG. 8 shows a third sectional view of the fourth exemplary embodiment of a measuring device according to the invention;

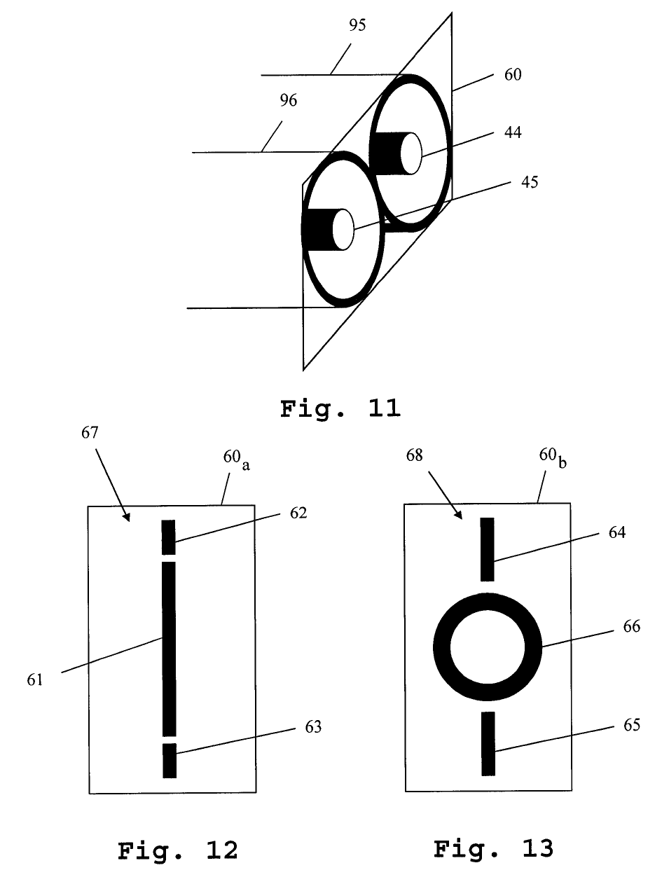

FIG. 9 shows a detail of a fifth exemplary embodiment of a measuring device according to the invention;

FIG. 10 shows a detail of a sixth exemplary embodiment of a measuring device according to the invention;

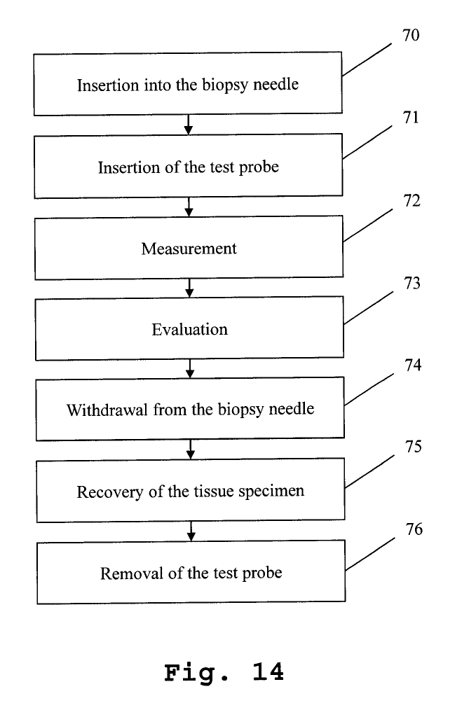

FIG. 11 shows a detail of a seventh exemplary embodiment of a measuring device according to the invention;

FIG. 12 shows a detail of an eighth exemplary embodiment of a measuring device according to the invention;

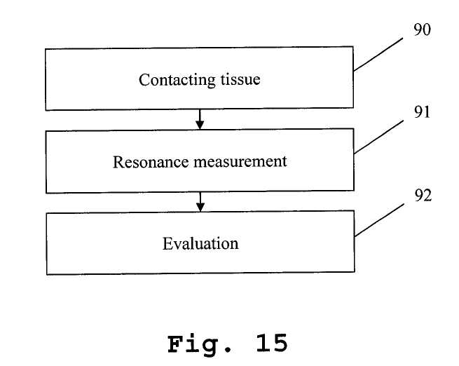

FIG. 13 shows a detail of a ninth exemplary embodiment of a measuring device according to the invention;

FIG. 14 shows a flow diagram of a first exemplary embodiment of the method according to the invention; and

FIG. 15 shows a flow diagram of a second exemplary embodiment of the method according to the invention.

Initially, with reference to FIG. 1, the general structure and the general functioning of the measuring device according to the invention is explained with reference to an exemplary embodiment. The structure and functioning of various forms of the measuring device according to the invention is shown with reference to FIGS. 2-13. With reference to FIG. 14 and FIG. 15, the functioning of the method according to the invention is then presented. The presentation and description of identical elements in similar drawings has not been repeated in some cases.

In FIG. 1, a first exemplary embodiment of a measuring device according to the invention is presented. A test probe 1 is connected by means of a connecting line 6 to a housing 5. The housing 5 contains a control device 4, a test-signal transmitter 2 and a test-signal receiver 3. The control device 4 is connected to the test-signal transmitter 2 and to the test-signal receiver 3. The control device 4 controls both the test-signal transmitter 2 and also the test-signal receiver 3.

The test probe 1 is brought into contact with a tissue to be investigated for the implementation of a measurement. This can be implemented both by surface contact and also by insertion. The control device 4 controls the test-signal transmitter 2 in such a manner that the latter transmits a test signal by means of the test probe into the tissue. The test signal is scattered by the tissue. Furthermore, the control device 4 controls the test-signal receiver 3 in such a manner that the latter receives the scattered test signal. The control device 4 evaluates the scattered test signal. In this context, it determines abnormalities of the tissue. Abnormalities are, for example, tumorous tissue changes. If an abnormality has been determined in the tissue at the location of the measurement, a tissue specimen is removed for further investigation by means of the test probe. In this context, the tissue removed largely agrees with the tissue, which has been tested with the test signal.

FIG. 2 shows a detail of a measuring device. In this view, the front-end of test probe 1 according to the invention is shown. This front-end consists of a coaxial line 7, one end of which is open. The coaxial line 7 here contains an inner conductor 12, a dielectric 13, an outer conductor 11 and an insulation 10. The dielectric 13 here is shown as transparent to allow a better view. The dielectric 13 fills the entire space between the inner conductor 12 and the outer conductor 11. The insulation 10 completely encloses the external side of the outer conductor 11.

In order to implement a measurement, the open end of the coaxial line 7 is brought into contact with the tissue to be investigated. As an alternative, a positioning in the proximity of the tissue to be investigated is possible. A test signal then impinges on the tissue by means of the coaxial line 7. The test signal scattered by the tissue is also received and rerouted by means of the coaxial line 7.

The insulation is used for reasons of biological compatibility. A bodily reaction to the material of the coaxial line is accordingly avoided.

However, because of the blunt end, this exemplary embodiment is suitable only for openly accessible tissue, because an insertion into the tissue is possible only with great difficulty if at all.

In FIG. 3, a first detail of a third exemplary embodiment of the measuring device according to the invention is presented. This exemplary embodiment corresponds largely to the exemplary embodiment presented in FIG. 2. However, here, the end of the coaxial line 8 is not blunt but provides an acute angle. The outer conductor 21 corresponds to the outer conductor 11 from FIG. 2. The dielectric 23 corresponds to the dielectric 13 from FIG. 2. The inner conductor 22 corresponds to the inner conductor 12 from FIG. 2.

With the test probe illustrated here, an insertion into the tissue is possible. Through the use of a low-friction material for the insulation 10, a simple insertion is guaranteed. However, the insulation 10 is not absolutely necessary. In the case of insensitive tissue types, it is not required. Also, an insulation is not necessary with the use of materials for the coaxial line, which provoke only slight reactions.

FIG. 4 shows a second detail of the third exemplary embodiment of a measuring device according to the invention. A biopsy needle 30 is formed by a hollow metal tube and provides an acute angle at its end. The edges of the end are sharp. An insertion of the biopsy needle 30 into tissue is accordingly possible. The internal diameter of the biopsy needle 30 in this context is slightly larger than the outer diameter of the insulation 10 from FIG. 3.

In FIG. 5, a third detail of the third exemplary embodiment of a measuring device according to the invention is illustrated. Here, the combination of the details shown in FIG. 3 and FIG. 4 is illustrated. Accordingly, the coaxial line 8 from FIG. 3 is introduced into the biopsy needle 30. The end of the coaxial line 8 and of the biopsy needle 30 are terminated in a flush manner.

The biopsy needle 30 gives the coaxial line 8 the necessary stability and the end of the coaxial line 8 the necessary sharpness for insertion into the tissue.

In order to implement a determination of tissue parameters with the test probe illustrated in this exemplary embodiment, the biopsy needle 30 with the coaxial line 8 disposed within it is initially inserted into the tissue. As an alternative, positioning on the surface of an already exposed tissue is possible. Once the desired location within the tissue has been reached, an electrical measurement is implemented by means of the coaxial line 8. If the need for a biopsy is inferred from the result of the electrical measurement, the coaxial line 8 is withdrawn within the biopsy needle 30 by the length of the desired tissue specimen. Following this, the biopsy needle is further inserted into the tissue by the desired length of the tissue specimen. In this context, the tissue specimen penetrates into the biopsy needle and is fixed by the latter. Finally, the test probe with the tissue specimen is withdrawn from the tissue. The tissue specimen is pressed out of the biopsy needle by pushing back the coaxial line 8.

FIG. 6 shows a first detail of a fourth exemplary embodiment of a measuring device according to the invention. The exemplary embodiment shows a test probe according to the invention in a sectional view. This test probe contains only one stable coaxial line 81. The coaxial line 81 here consists of an outer conductor 21, a dielectric 80 and an inner conductor 22. The outer conductor 21 here is designed to be more stable than in the case of a conventional coaxial line. Accordingly, the outer conductor 21 stabilises the entire coaxial line 81 and ensures that no further stabilising components are required. Additionally, a stable design of the inner conductor 22 is optionally possible. The dielectric 80 in this context is mobile relative to the outer conductor 21 and the inner conductor 22. To allow a better view, the insulation, which surrounds the outer conductor, is not illustrated.

In order to determine tissue parameters, the test probe is inserted into the tissue. When the front end of the test probe has reached a point within the tissue to be evaluated, a measurement of the electrical parameters of the tissue is implemented. For this purpose, a test signal impinges on the tissue by means of the coaxial line 81. The tissue scatters the test signal. The scattered test signal is also received by the coaxial line 81 and routed for further processing.

If given tissue parameters, which make a biopsy seem necessary, are determined by the further processing, a tissue specimen is removed. This will be described in greater detail with reference to FIG. 7-FIG. 8.

In order to remove a tissue specimen, while the end of the coaxial line 81 touches the tissue to be investigated, the dielectric 80 is withdrawn by at least the length of the tissue specimen to be removed. Accordingly, in FIG. 7, a second detail of the fourth exemplary embodiment of a measuring device according to the invention is presented. This presentation corresponds largely to that of FIG. 6. However, the dielectric 80 here has been withdrawn by comparison with the outer conductor 21 and the inner conductor 22 relative to the front-end of the test probe.

After the dielectric 80 has been withdrawn, the test probe is inserted further into the tissue by at least the length of the tissue specimen to be removed. In this context, the tissue penetrates into the intermediate space between the outer conductor and inner conductor. As an alternative, the withdrawal of the dielectric 80 can be implemented at the same time as the further insertion of the test probe into the tissue. FIG. 8 shows a third detail of the fourth exemplary embodiment of a measuring device according to the invention. In this detail, the test probe is presented with a removed tissue specimen 82. The tissue specimen 82 here is fixed by the outer conductor 21 and the inner conductor 22. In this manner, a loss of the tissue specimen 82 during the withdrawal of the test probe from the tissue is avoided.

To remove the tissue specimen 82, after the withdrawal of the test probe from the tissue, the dielectric is pushed back to its original place illustrated in FIG. 6 relative to the outer conductor 21 and the inner conductor 22. The tissue specimen 82 is then ejected at the front-end of the test probe.

In FIG. 9, a detail of a fifth exemplary embodiment of a measuring device according to the invention is presented. This view shows the front-end of an alternative test probe. The test probe contains two coaxial lines 48, 49. The coaxial lines each provide an outer conductor 40, 41, a dielectric 46, 47 and an inner conductor 44, 45. The dielectric 46, 47 has been illustrated as transparent here. To allow a better view, the insulation, which surrounds each individual coaxial line, is not shown. The front-end of the coaxial lines 48, 49 here is blunt.

This test probe corresponds to the test probe illustrated in FIG. 1. That is to say, a deep insertion of the test probe into the tissue is not possible. Only a surface positioning on a tissue and the removal of a tissue specimen from the tissue surface is possible.

However, more accurate measurements of the electrical parameters of the tissue can be implemented with this alternative test probe. Accordingly, a test signal is transmitted through the first coaxial line 48 into the tissue. The scattered test signal is conducted via the second coaxial line 49 for further processing. In this manner, transmission measurements can be implemented alongside reflection measurements.

FIG. 10 shows a detail of a sixth exemplary embodiment of the measuring device according to the invention. This view also shows the front end of an alternative test probe. The test probe contains two coaxial lines 58, 59. The coaxial lines 58, 59 contain an outer conductor 50, 51, a dielectric 56, 57, an inner conductor 54, 55 and an insulation 52, 53. The front ends of the coaxial lines 58, 59 here are designed in a bevelled manner and form a combined tip, which provides an acute angle.

To implement a determination of tissue parameters, the two coaxial lines 58, 59 are introduced into a biopsy needle in a similar manner to the exemplary embodiment shown in FIG. 4. The biopsy needle is of oval or kidney-shaped cross-section by contrast with a conventional biopsy needle.

As an alternative, the design of the two coaxial lines 58, 59 with sharp front edges, a stable design of the outer conductor 50, 51 and mobile dielectrics 56, 57 similar to the exemplary embodiment illustrated in FIG. 6-FIG. 8 is possible.

In FIG. 11, a detail of a seventh exemplary embodiment of the measuring device according to the invention is presented. The exemplary embodiment presented here is a modification of the exemplary embodiment shown in FIG. 9. A printed-circuit board 60 is attached in front of the open ends of the coaxial lines 95, 96. The printed-circuit board 60 has been illustrated as transparent to allow better visibility. The further design of the printed-circuit board 60 and its function will be explained in greater detail with reference to FIG. 12-FIG. 13.

FIG. 12 shows a detail of an eighth exemplary embodiment of a measuring device according to the invention. Here, a first alternative of the printed-circuit board 60 from FIG. 11 is illustrated. The printed-circuit board 60ₐ provides on its side facing away from the coaxial lines 48, 49 a resonator circuit 67 consisting of three strip conductors 61, 62, 63. The first two strip conductors 62, 63 in this context provide only a short length. The third strip conductor 61 provides a substantially longer length. The strip conductors 50, 62, 63 are arranged in one line here, largely centrally on the printed-circuit board 60ₐ. The first two strip conductors 62, 63 in this context are connected to the inner conductors 44, 45. This connection is implemented by means of a through contact through the printed-circuit board 60ₐ. The third strip conductor 61 is connected in a non-conducting manner to the inner conductors 44, 45 or to the other strip conductors 62, 63. However, the distances between the third strip conductor 61 and the other strip conductors 62, 63 are small. A capacitive coupling occurs. The third strip conductor 61 forms a resonator here. The resonance wavelength of the resonator in the exemplary embodiment is approximately double its length. It is therefore a λ/2 resonator. The precise resonance wavelength is dependent upon the surroundings of the resonator. Particularly accurate measurements are achieved with a conductive coating of the rear side of the printed-circuit board 60ₐ. This rear-side coating provides recesses at the through passages of the inner conductor 44, 45 and of the dielectric 46, 47. The recesses preferably provide at least the diameter of the coaxial lines 95, 96.

To implement a determination of electrical tissue parameters, the printed-circuit board is brought into contact with tissue to be investigated. As an alternative, it is sufficient to bring the printed-circuit board into the proximity of the region under investigation. In this context, the tissue under investigation is brought into the proximity of the resonator or respectively into contact with the resonator. This changes the properties of the surroundings of the resonator. This influences the resonance wavelength of the resonator. By means of the first coaxial line 95, a test signal impinges on the resonator. By means of the second coaxial line 96, the test signal influenced by the resonator is conducted for further processing. The exact resonance frequency, any losses occurring and the shape of the resonance curve of the resonator can be determined in this manner. Electrical tissue parameters are determined on the basis of the given parameters.

In FIG. 13, a detail of a ninth exemplary embodiment of a measuring device according to the invention is presented. In this exemplary embodiment, an alternative form of the resonator is presented. The strip conductors 64, 65, 66 form the resonator circuit 68. The third strip conductor 66 here is designed in a circular shape. The other strip conductors 64, 65 correspond to the other strip conductors 62, 63 from FIG. 12. The first strip conductor 66 here also acts as a resonator. In this context, the circumference of the circular strip conductor 66 is approximately half the resonance wavelength of the resonator. Here also, the exact resonance frequency of the resonator is determined by the surrounding tissue.

A removal of a tissue specimen is also possible with a test probe according to this exemplary embodiment. For this purpose, the printed-circuit board 60 is designed to be very small. Furthermore, the coaxial lines are arranged within a biopsy needle in a similar manner to the exemplary embodiment shown in FIG. 5. The removal is implemented, as shown there, after the withdrawal of the coaxial lines with the printed-circuit board 60.

FIG. 14 shows a first exemplary embodiment of the method according to the invention. In a first step 70, a coaxial line open at one end is pushed into a biopsy needle. Together, they form a test probe. In a second step 71, the test probe is inserted into a tissue, wherein the tip of the biopsy needle and therefore also the end of the coaxial line come to be disposed at a location to be investigated within the tissue. In a third step 72, a measurement of electrical parameters of the tissue is implemented. For this purpose, a test signal impinges on the tissue. The test signal is scattered by the tissue. The scattered test signal is received. In a fourth step 73, the received test signal is evaluated.

By comparing the transmitted test signal with the received test signal, electrical parameters of the tissue are determined. For example, the dielectric constants, are determined, of which the characteristic is determined via the frequency and the conductivity of the tissue at the location to be investigated. If the determined tissue parameters give cause for further investigations, in a fifth step 74, the coaxial line is withdrawn from the biopsy needle by at least the length of a tissue specimen to be removed. In a sixth step 75, the test probe is inserted further into the tissue by at least the length of the tissue specimen to be removed. The tissue now penetrates into the biopsy needle and is fixed by the latter. In a final seventh step 76, the test probe is withdrawn from the tissue together with the tissue specimen.

For determining electrical parameters of the tissue, a second exemplary embodiment of the method according to the invention, as shown in FIG. 15, can be used as an alternative. In a first step 90, a tissue to be investigated is contacted with a test probe. In this context the test probe contains a resonator. The resonance properties of the resonator here are influenced by the tissue. That is to say, the resonance wavelength of the resonator changes dependent upon the properties of the tissue. In a second step 91, a resonance measurement is implemented. For this purpose, a plurality of test signals of different frequency impinge in succession on the resonator. The resulting signal of the resonator is then measured and rerouted for evaluation. In a third step 92, through comparison of the transmitted test signal and the signal received from the resonator, the resonance wavelength of the resonator is determined. From the resonance wavelength, inferences can be made regarding the properties of the tissue, especially in cases of breast cancer or prostate cancer. The determination of the electrical parameters can be implemented using a network analyser.

The tissue under investigation can be dead or living human, animal or plant tissue. In particular, it is possible to draw inferences from the tissue parameters regarding the presence of tumorous changes of the tissue.

The invention is not restricted to the exemplary embodiment illustrated. Accordingly, different types of tissue can be investigated. A use for material testing is also conceivable. All of the features described above or features illustrated in the drawings can be advantageously combined with one another as required within the framework of the invention.

1. A measuring device with a control device, a test-signal transmitter, a test-signal receiver and a test probe,
wherein the test probe contains at least one coaxial line,
wherein the control device controls the test-signal transmitter in such a manner that it transmits the test signal by means of the coaxial line into a given location of a tissue,
wherein the test signal is scattered by the tissue,
wherein the control device controls the test-signal receiver in such a manner that it receives the scattered test signal,
wherein the control device evaluates the received test signal,
wherein the test probe is designed in such a manner that, a tissue specimen can be removed by it at the given location of the tissue.

2. The measuring device according to claim 1, characterized in that
the test probe contains a biopsy needle, and that the coaxial line is disposed within the biopsy needle.

3. The measuring device according to claim 2, characterized in that
the coaxial line is movable within the biopsy needle,
that the coaxial line can be withdrawn by at least a length of the tissue specimen at a location, at which a tissue specimen is to be removed,
and that the tissue specimen can penetrate into the biopsy needle and can be fixed by the latter.

4. The measuring device according to claim 1, characterized in that
an end of the coaxial line provides sharp edges, and
that the coaxial line provides a rigid form.

5. The measuring device according to claim 4, characterized in that
the coaxial line contains an inner conductor, an outer conductor and a dielectric,
that the dielectric is movable,
that the dielectric can be withdrawn at a location within a tissue, at which a tissue specimen is to be removed, by at least one length of the tissue specimen, and
that the tissue specimen can penetrate into an intermediate cavity between the outer conductor and the inner conductor and can be fixed by the latter.

6. The measuring device according to claim 1, characterized in that
the test probe contains two coaxial lines,
that the test-signal transmitter transmits the test signal by means of a first coaxial line into the tissue, and
that the test-signal receiver receives the test signal by means of a second coaxial line.

7. A measuring device with a control device, a test-signal transmitter, a test-signal receiver and a test probe,
wherein the test probe contains at least two coaxial lines and a resonator circuit,
wherein a first coaxial line is connected to the resonator circuit and to the test-signal transmitter,
wherein a second coaxial line is connected to the resonator circuit and to the test-signal receiver,
wherein the control device controls the test-signal transmitter in such a manner that it transmits a test signal by means of the first coaxial line to the resonator circuit,
wherein the control device controls the test-signal receiver in such a manner that it receives the test signal modified by the resonator circuit from the resonator circuit by means of the second coaxial line,
wherein the resonance properties of the resonator circuit are influenced by tissue disposed in their proximity, and
wherein the control device evaluates the received test signal.

8. The measuring device according to claim 7, characterized in that
the resonator circuit consists of a printed-circuit board with at least one strip conductor disposed on it.

9. The measuring device according to claim 8, characterized in that
three strip conductors are disposed on the printed-circuit board,
that a first strip conductor is connected in a conducting manner to the first coaxial line,
that a second strip conductor is connected in a conducting manner to the second coaxial line,
that a third strip conductor is disposed between the first strip conductor and the second strip conductor, and that the third strip conductor is connected in a capacitive manner to the first strip conductor and to the second strip conductor.

10. The measuring device according to claim 9, characterized in that
the third strip conductor is annular or rectilinear in shape, that the third strip conductor determines the resonance wavelength of the resonator circuit, and
that the length of the third strip conductor is ¼ to ¾, preferably ½ of the resonance wavelength of the resonator circuit.

11. The measuring device according to claim 8, characterized in that
tissue to be investigated is placed in the proximity of the third strip conductor.

12. The measuring device according to claim 7, characterized in that
the test probe is designed in such a manner that a tissue specimen can be removed by it at a given location of the tissue.

13. A method for determining tissue parameters with a control device, a test-signal transmitter, a test-signal receiver and a test probe,
wherein an end of the test probe is brought into the proximity of a tissue to be investigated,
wherein the test-signal transmitter is controlled in such a manner by the control device (4), that it transmits a test signal into a given location of the tissue,
wherein the test signal is scattered by the tissue,
wherein the test-signal receiver is controlled in such a manner by the control device, that it receives the scattered test signal,
wherein the received test signal is evaluated by the control device, and
wherein the test probe removes a tissue specimen at a given location of the tissue.

14. The method according to claim 13, characterized in that
the test probe contains a biopsy needle and at least one coaxial line, and that the following steps are implemented:
complete insertion of the coaxial line into the biopsy needle;
insertion of the test probe into the tissue, wherein a tip of the test probe is brought to the given location;
impinging of the test signal on the tissue;
reception of the scattered test signal
determination of the electrical tissue parameters
withdrawal of the coaxial line from the test probe by at least one length of the tissue specimen;
recovery of the tissue specimen;
fixing of the tissue specimen by the biopsy needle; and
removal of the test probe.

15. The method according to claim 13, characterized in that
the test probe contains at least one coaxial line,
that one end of the coaxial line provides sharp edges,
that the coaxial line provides a rigid form,
that the coaxial line contains an inner conductor, an outer conductor and a dielectric,
that the dielectric is movable relative to the inner conductor and the outer conductor,
that the following steps are implemented:
complete insertion of the dielectric into the coaxial line;
insertion of the test probe into the tissue, wherein the tip of the test probe is brought to the given location;
impinging of the test signal on the tissue;
reception of the scattered test signal;
determination of the electrical tissue parameters;
withdrawal of the dielectric from the coaxial line by at least a length of the tissue specimen;
recovery of the tissue specimen;
fixing of the tissue specimen by the inner conductor and the outer conductor, and
removal of the test probe.

16. A method for determining tissue parameters with a control device, a test-signal transmitter, a test-signal receiver and a test probe,
wherein the test probe contains at least two coaxial lines and a resonator circuit,
wherein the resonator circuit disposed at one end of the test probe is brought into the proximity of a tissue to be investigated,
wherein the test-signal transmitter is controlled by the control device in such a manner that a test signal is transmitted by it by means of the first coaxial line to the resonator circuit,
wherein the test-signal receiver is controlled by the control device in such a manner that the test signal modified by the resonator circuit is received by it by means of the second coaxial line from the resonator circuit,
wherein the resonance properties of the resonator circuit are influenced by tissue disposed in its proximity, and
wherein the received test signal is evaluated by the control device.

17. The method according to claim 16, characterized in that
the test probe removes a tissue specimen at the given location of the tissue.
